(19) United States
(12) Patent Application Publication
(10) Pub. No.: US 2020/0367770 A1
(43) Pub. Date: Nov. 26, 2020

(54) BLOOD PRESSURE MEASUREMENT DEVICE WITH A MEMS PUMP AND CONTROL METHOD FOR THE SAME

(71) Applicant: MICROLIFE CORPORATION, Taipei City (TW)

(72) Inventor: Chia-Ming LIN, Taipei City (TW)

(73) Assignee: MICROLIFE CORPORATION

(21) Appl. No.: 16/986,375

(22) Filed: Aug. 6, 2020

Related U.S. Application Data
(63) Continuation of application No. 15/802,466, filed on Nov. 3, 2017.

(30) Foreign Application Priority Data
Nov. 11, 2016 (TW) 105136775

(51) Int. Cl. A61B 5/022; H01L 41/04; A61B 5/00; F04B 17/00

(52) U.S. Cl. CPC A61B 5/02233; H01L 41/042; A61B 5/02141; F04B 17/003; A61B 5/742

(57) ABSTRACT

This invention discloses a blood pressure measurement device including a cuff having a bladder wrap around an object to be measured, a MEMS pump for inflating the bladder with air, a pressure sensor for monitoring the pressure in the bladder, and a microcontroller for controlling the pressure sensor to continuously receive pressure signals during the process of inflating the bladder, converting the pressure signals into blood pressure values, and controlling a drive voltage level of the MEMS pump to maintain an air inflation speed of the MEMS pump in a predetermined inflation speed range. The invention further discloses a method for controlling a fixed frequency change of the blood pressure measurement device.

### CROSS-REFERENCE TO RELATED APPLICATIONS

This application is a continuation of U.S. application Ser. No. 15/802,466 filed on Dec. 3, 2017, which is a non-provisional application claims priority under 35 U.S.C. § 119(a) on Patent Application No(s). 105136775 filed in Taiwan, R.O.C. on Nov. 11, 2016, the entire contents of which are hereby incorporated by reference.

### FIELD OF INVENTION

The present invention relates to a blood pressure measurement device, in particular, to a blood pressure measurement device with a MEMS pump. The present invention also relates to a control method of the blood pressure measurement device, in particular, to a control method for controlling the inflation speed of the MEMS pump in a predetermined range during the inflation process of the bladder.

### BACKGROUND OF INVENTION

### 1. Description of the Related Art

In general, there are mainly two ways for the conventional blood pressure measurement device to measure blood pressure: deflation type measurement, and inflation type measurement. In a deflation type blood pressure measurement process, a pump is provided for inflating the bladder in the cuff wrapped around a user's arm or wrist, so that the bladder is inflated to a predetermined pressure to press the user's artery. When the air in the bladder is released, the bladder is deflated. During the deflation stage, a pressure sensor is provided for detecting pulse pressure caused by vasoconstriction, and such pulse pressure is converted into a blood pressure value. In the inflation type blood pressure measurement process, the blood pressure is measured during the inflation process.

Recently, the technology of micro-electromechanical systems (MEMS) pump is becoming more matured, and MEMS pumps are used extensively in blood pressure measurement devices. MEMS pump made of piezoelectric material with anti-piezoelectric effect has the features of low noise, high control precision, and stable output. Blood pressure measurement device comprising MEMS pump is capable of capturing the pulse pressure caused by vasoconstriction during the inflation process of the bladder, and obtaining a blood pressure value by analyzing, computing and converting the pulse pressure.

However, it is necessary to improve the accuracy of the blood pressure measurement device having the MEMS pump. It is also desirable to improve and the stability of the inflation in order to achieve a stable inflation speed. In the inflation process, it is necessary to control the drive voltage or drive frequency of the MEMS pump via a microcontroller to maintain slow and steady rise of the pressure in the bladder, so as to maintain a stable inflation speed. Therefore, a complicated control circuit is required, and a good effect is usually difficult to achieve.

### 2. Summary of the Invention

In view of the aforementioned drawbacks, it is a primary objective of the present invention to overcome the drawbacks of the prior art by providing a blood pressure measurement device to control the drive voltage level of a MEMS pump, so that the inflation ability of the MEMS pump can be changed timely to maintain a stable pressurization speed for the inflation of the bladder, or to maintain a stable air flow in the bladder.

To achieve the aforementioned and other objectives, the present invention provides a blood pressure measurement device with a MEMS pump, and the device comprises a cuff having a bladder, a MEMS pump, and a microcontroller. The cuff is wrapped around an object to be measured. The MEMS pump is for inflating the bladder with air. The microcontroller controls the drive voltage level of the MEMS pump to control the air inflation speed of the MEMS pump within a predetermined inflation speed range.

The microcontroller of the blood pressure measurement device of the present invention controls the drive voltage level of the MEMS pump using a voltage regulator circuit through a motor driving control circuit.

The microcontroller of the blood pressure measurement device of the present invention emits a fixed-frequency signal to the motor driving control circuit to provide a fixed drive frequency of the MEMS pump.

The blood pressure measurement device of the present invention further comprises a motor driving control circuit for issuing a fixed-frequency signal to provide a fixed drive frequency of the MEMS pump.

In the blood pressure measurement device of the present invention, the predetermined inflation speed range is from 4 to 6 mmHg/sec.

The blood pressure measurement device of the present invention further comprises a pressure sensor for monitoring the pressure in the bladder during the process of inflating the bladder with air.

In the blood pressure measurement device of the present invention, the MEMS pump is further provided for releasing air from the bladder.

The present invention also provides a control method for the blood pressure measurement device having the MEMS pump, wherein the blood pressure measurement device comprises a cuff having a bladder, which wraps around an object to be measured; a MEMS pump coupled to the bladder for inflating the bladder with air; a pressure sensor coupled to the bladder for monitoring the pressure in the bladder; and a microcontroller for receiving a plurality of pressure signals from the pressure sensor during the inflation process of the MEMS pump; and the control method comprises the steps of: (a) providing a fixed drive frequency and a drive voltage level to the MEMS pump for continuously inflating the bladder with air; (b) determining an inflation speed of the MEMS pump by the microcontroller according to the plurality of pressure signals provided by the pressure sensor, and if the inflation speed is greater than a predetermined inflation speed range, then the microcontroller will lower the drive voltage level, so that the inflation speed is controlled to the predetermined inflation speed range; and (c) converting the plurality of pressure signals into a blood pressure value using the microcontroller.

The present invention also provides another control method for the blood pressure measurement device with the MEMS pump, wherein the blood pressure measurement device comprises a cuff having a bladder, which wraps around an object to be measured; a MEMS pump coupled to the bladder for inflating the bladder with air; a pressure sensor coupled to the bladder for monitoring the pressure in the bladder; and a microcontroller for receiving a plurality of pressure signals from the pressure sensor during an inflation process of the MEMS pump; and the control method comprises the steps of: (a) providing a fixed drive frequency and a drive voltage level to the MEMS pump for continuously inflating the bladder with air; (b) determining an inflation speed of the MEMS pump using the microcontroller according to the plurality of pressure signals provided by the pressure sensor, and if the inflation speed is smaller than a predetermined inflation speed range, then the microcontroller will increase the drive voltage level, so that the inflation speed is increased to the predetermined inflation speed range; and (c) converting the plurality of pressure signals into a blood pressure value using the microcontroller.

The aforementioned control method of the present invention further comprises the step of detecting the pulse provided by the pressure sensor by the microcontroller after Step (a), and deflating the bladder if there is no pulse.

### BRIEF DESCRIPTION OF THE DRAWINGS

FIG. 1 is a perspective view of a blood pressure measurement device in accordance with the present invention;

FIG. 2 is a block diagram showing the components of a blood pressure measurement device in accordance with a first preferred embodiment of the present invention;

FIG. 3 is a graph of pressure versus time of a bladder of a blood pressure measurement device of the present invention;

FIG. 4 is a graph of the voltage versus time of a MEMS pump of a blood pressure measurement device of the present invention;

FIG. 5 is a block diagram showing the components of a blood pressure measurement device in accordance with a second preferred embodiment of the present invention; and

FIG. 6 is a flow chart of controlling a blood pressure measurement device of the present invention.

### DESCRIPTION OF THE PREFERRED EMBODIMENTS

To make it easier for our examiner to understand the objective, technical characteristics, structure, innovative features, and performance of the invention, we use preferred embodiments together with the attached drawings for the detailed description of the invention. For simplicity and clarity, the drawings are provided for showing the overall structure of the invention, and the characteristics of the prior art and their corresponding detailed description is omitted to avoid unnecessarily blurring the claims of the present invention. It is noteworthy that same numerals are used to represent the same elements in the drawings respectively.

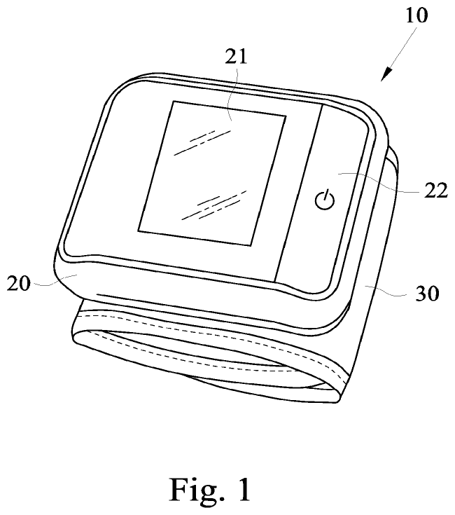

With reference to FIG. 1 for a perspective view of a blood pressure measurement device 10 in accordance with a preferred embodiment of the present invention, the blood pressure measurement device 10 of this preferred embodiment is a wrist sphygmomanometer. However, the invention is not limited to the wrist sphygmomanometer only, and people having ordinary skill in the art should understand that the blood pressure measurement device 10 may be an upper arm type sphygmomanometer or any other equivalent blood pressure measurement device. The blood pressure measurement device 10 comprises a main body 20 and a cuff 30 having a bladder (not shown in FIG. 1) and coupled to the main body 20. The main body 20 further has a display unit 21 and a user interface 22 such as a push button. The display unit 21 is provided for the users to observe operation information such as time, body temperature, local temperature, and humidity . . . etc., as well as measured blood pressure values. However, the type and function of the display unit of the blood pressure measurement device of the present invention are not limited. For example, a touch screen is also applicable for the present invention. In another preferred embodiment, the casing of the main body 20 may be a transparent or translucent casing, and its interior may have an alternating light source or a two-color light source, so that the colors may change with the pulse rate during the measurement; or a red or green light may be shown to indicate a high blood pressure or a standard blood pressure respectively.

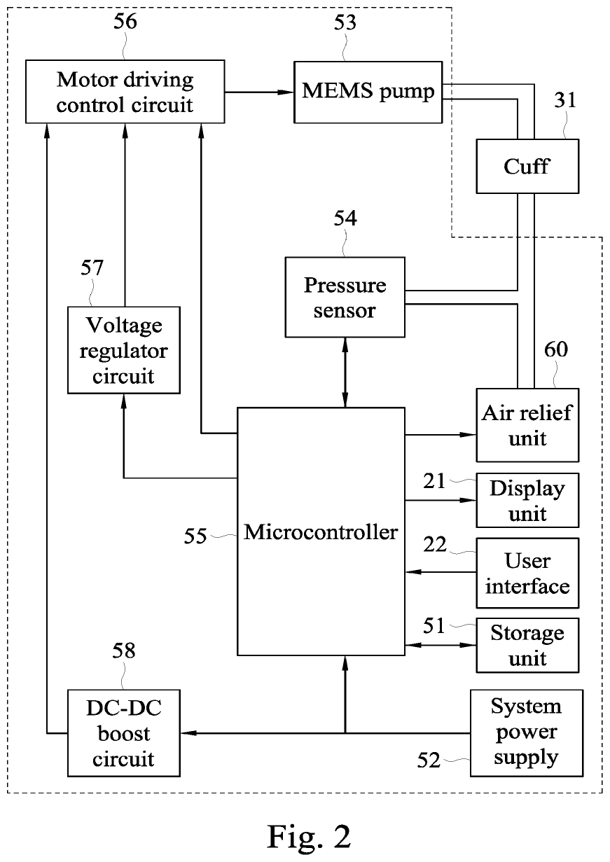

With reference to FIG. 2 for a block diagram showing the components in a main body 20 of a blood pressure measurement device 10 in accordance with the first preferred embodiment of the present invention (within the range which is indicated by the dotted line in the figure), a user starts the blood pressure measurement device 10 through a user interface such as a push button of the main body 20 to measure the user's blood pressure, and a system power supply 52 starts supplying electric power to the whole system. In this preferred embodiment, the system power supply 52 may be a battery or an external power supply such as a 110 VAC power supplied by a transformer. The pressure sensor 54 is coupled to a bladder 31 of the cuff for monitoring the pressure in the bladder 31 anytime, and sending a plurality of detected pressure signals to a microcontroller 55. People having ordinary skill in the art should understand that the pressure sensor 54 may be designed to be connected to a circuit which is communicated to the bladder 31 for detecting the pressure. In addition, the system power supply 52 also supplies electric power to a DC-DC boost circuit 58 which is used for boosting the DC voltage of the system power supply 52 to a DC voltage applicable for the motor driving control circuit 56.

The microcontroller 55 controls a voltage regulator circuit 57 to supply an initial voltage V0 (preferably 10 volts) to the motor driving control circuit 56 at an initialization stage; meanwhile the microcontroller 55 drives the motor driving control circuit 56 via the voltage regulator circuit 57 to carry out a constant-speed inflation of the MEMS pump 53. The microcontroller 55 also emits a pulse width modulation (PWM) fixed-frequency signal to the motor driving control circuit 56 to provide a fixed drive frequency to the MEMS pump 53. In other words, a constant frequency is maintained in the whole inflation process. It is noteworthy that the voltage regulator circuit 57 can adjust the drive voltage level of the MEMS pump by changing the resistance or current, so as to achieve the constant-speed inflation effect of the MEMS pump 53.

The MEMS pump 53 starts inflating the bladder 31 according to the predetermined values of the initial voltage V0 and the constant drive frequency. In the meantime, the pressure sensor 54 is controlled by the microcontroller 55, so that the pressure in the bladder is detected once for a certain period of time (preferably once for every 0.5 second) during the inflation process, and a plurality of pressure signals having the pressure values are sent continuously to the microcontroller 55.

The microcontroller 55 determine the inflation speed of the MEMS pump 53 based on the pressure values of the pressure signals at minimum of two different time, meanwhile the microcontroller 55 determines whether or not the current inflation speed is maintained within a predetermined inflation speed range (which is stored in a storage unit 51). When the current inflation speed is greater than the predetermined inflation speed range, the microcontroller 55 will adjust an input/output (I/O) pin of the voltage regulator circuit 57 and use the motor driving control circuit 56 to lower the drive voltage level of the MEMS pump 53, so as to control the inflation speed and return the current inflation speed to its predetermined inflation speed range. When the current inflation speed is smaller than the predetermined inflation speed range, the microcontroller 55 will adjust the input/output (I/O) pin of the voltage regulator circuit 57 and use the motor driving control circuit 56 to increase the drive voltage level of the MEMS pump 53, so as to increase the current inflation speed and allow the current inflation speed to reach its predetermined inflation speed range. In this preferred embodiment, the predetermined inflation speed range is from 2 to 7 mmHg/sec, preferably 4 to 6 mmHg/sec. In addition, the storage unit 51 is a memory, but the present invention is not limited to such arrangement only. The storage unit 51 is provided for storing at least one record of the blood pressure values. In another preferred embodiment, the storage unit 51 further stores user's related data, and a user interface is provided for switching and displaying the identity information or physiological information on the aforementioned display unit 21. People having ordinary skill in the art should understand that the user related data may be stored in the storage unit 51 by an external electronic device via a wireless or cable transmission.

The microcontroller 55 analyzes and computes a plurality of pressure signals obtained by the pressure sensor 54, converts the pressure signals into blood pressure values, and displays the blood pressure values on the display unit 21. Since such conversion process is well known, it will not be described here.

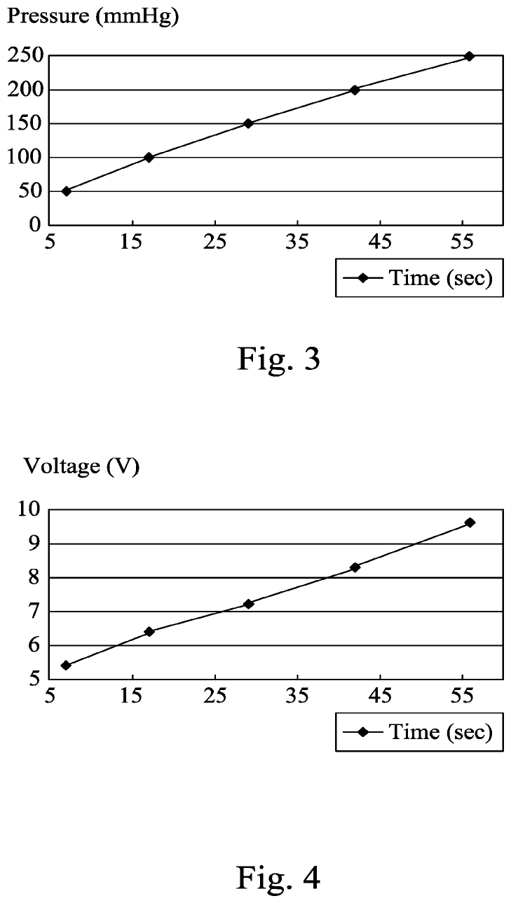

With reference to FIG. 3 for a graph of pressure versus time of the blood pressure device in accordance with the present invention, the control method of the blood pressure measurement device 10 of the present invention restricts the inflation speed in a specific range and carries out the inflation at a substantially constant speed in order to record the amplitude of the pulses more precisely, and the microcontroller 55 can analyze and compute the blood pressure values more accurately.

With reference to FIG. 4 for a graph of drive voltage versus time of the MEMS pump 53 in accordance with the present invention, the drive voltage level of the MEMS pump 53 is adjusted continuously with time. The voltage level is not just increased according to a linear control only, but the voltage level is also changed continuously to maintain the inflation speed in a predetermined inflation speed range.

In another preferred embodiment of the present invention, the blood pressure measurement device further comprises a flow sensor for detecting the amount of inflation to replace the function of the pressure sensor for monitoring the pressure in the bladder. Therefore, the aforementioned flow sensor continuously transmits current flow signal to the microcontroller 55, so that the microcontroller 55 continuously adjusts the drive voltage level of the MEMS pump 53 to maintain the inflation speed in a predetermined inflation speed range.

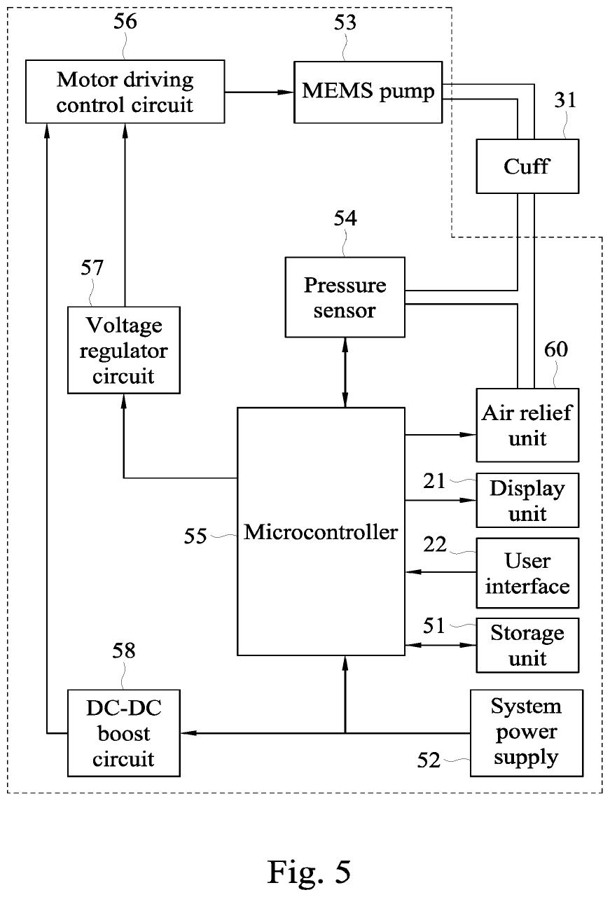

With reference to FIG. 5 for the block diagram showing the components of a blood pressure measurement device in accordance with the second preferred embodiment of the present invention, this preferred embodiment is substantially the same as the first preferred embodiment. Their difference resides on that the motor driving control circuit 56 of this embodiment emits a pulse width modulation (PWM) fixed-frequency signal, preferably a self-feedback signal to provide a fixed drive frequency to the MEMS pump 53. In other words, the frequency is maintained constant during the whole inflation process. The microcontroller 55 adjusts the duty ratio of the voltage regulator circuit 57 to change the drive voltage level of the MEMS pump 53.

In details, the microcontroller 55 converts and computes the current inflation speed of the MEMS pump 53 according to the pressure values of the pressure signals at minimum of two different time, meanwhile the microcontroller 55 determines whether or not the current inflation speed is maintained in a predetermined inflation speed range (which is stored in the storage unit 51). When the current inflation speed is greater than a predetermined inflation speed range, the motor driving control circuit 56 provides a pulse width modulation (PWM) fixed-frequency signal. The microcontroller 55 adjusts and outputs the fixed-frequency duty ratio to the voltage regulator circuit 57. As a result, different duty ratios are outputted so that the voltage regulator circuit 57 produces different voltage levels; and the drive voltage of the MEMS pump 53 becomes smaller. In this way, the inflation speed can be controlled in the predetermined inflation speed range. When the current inflation speed is smaller than the predetermined inflation speed range, the adjusted drive voltage becomes greater to increase the inflation speed and return the current inflation speed to its predetermined inflation speed range.

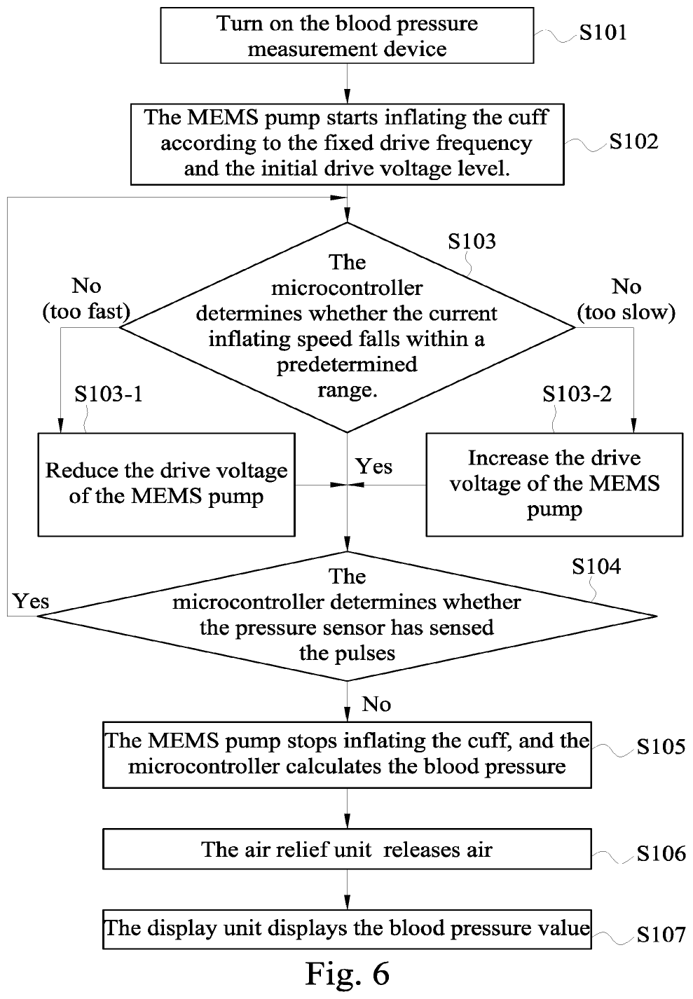

With reference to FIG. 6 for the flow chart of controlling the blood pressure measurement device 10 in accordance with the first or second preferred embodiment of the present invention. The present invention uses the inflation type measurement process which measures the blood pressure in the inflation process for illustrating the invention. The method for controlling the blood pressure measurement device 10 includes the following steps:

When a user presses a push button 22 to turn on the blood pressure measurement device 10 (Step S101), the MEMS pump 53 starts inflating the bladder 31 with air according to the predetermined values of the fixed drive frequency and the initial voltage level (Step S102). The microcontroller 55 determines whether or not the current inflation speed of the MEMS pump 53 falls within a predetermined inflation speed range (Step S103). If the current inflation speed is greater than the predetermined inflation speed range, then the drive voltage level of the MEMS pump 53 will be lowered to return the inflation speed to its predetermined inflation speed range (Step S103-1). On the other hand, if the current inflation speed is smaller than the predetermined inflation speed range, then the drive voltage of the MEMS pump 53 will be increased to return the inflation speed to its predetermined inflation speed range (Step S103-2).

As shown in FIGS. 3 and 6, no matter whether the current inflation speed of the MEMS pump 53 falls within the predetermined range or not, the microcontroller 55 will determine whether a pulse is generated from the artery and caused by a change of pressure detected by the pressure sensor 54 in order to determine whether the blood pressure measurement is completed (Step S104). If no pulse is detected, then the MEMS pump 53 will stop the inflation, and the microcontroller 55 will calculate the blood pressure value (Step S105). It is noteworthy that after the MEMS pump 53 starts the pressurization for the inflation, the microcontroller will continue to detect the pulse via the pressure sensor 54. Next, an air relief unit 60 installed in the main body 20 releases air from the bladder after step S105 (Step S106). And finally, the calculated blood pressure value is displayed on the display unit 21. Although the blood pressure value is displayed in Step S107 after the air relief unit 60 releases air, the present invention is not limited to such arrangement (Step S107). The step of displaying the blood pressure value on the display unit 21 may take place before the air relief unit 60 releases the air. As long as the MEMS pump 53 has stopped the inflation process and the microcontroller 55 has converted the plurality of pressure signals received by the pressure sensor 54 into the blood pressure values, the blood pressure values may be displayed on the display unit 21. In another preferred embodiment, the MEMS pump 53 may release air in an opposite direction. In other words, the MEMS pump 53 replaces the air relief unit 60 to release the air from the bladder quickly without applying any drive voltage.

Although the control method of the present invention is illustrated by the first and second preferred embodiments, the control method may also be applied to another embodiment by using a flow sensor to replace the pressure, and their only difference resides on that the microcontroller of the first or second preferred embodiment monitors the pressure in the bladder to determine the inflation speed. The microcontroller also controls and regulates the voltage level of the MEMS pump. In the embodiment which the flow sensor is adapted, the microcontroller determines the inflation speed by monitoring the flow rate of the fluid during the inflation process so as to control and regulate the voltage level of the MEMS pump.

What is claimed is:

1. A blood pressure measurement device with a MEMS pump, comprising:
a cuff having a bladder for wrapping around an object to be measured;
a MEMS pump for inflating and deflating the bladder with air respectively during an inflation process and a deflation process; and
a microcontroller controlling a DC drive voltage level of the MEMS pump so as to maintain a constant air inflation speed of the MEMS pump;
wherein the microcontroller controls the DC drive voltage level of the MEMS pump by a voltage regulator circuit through a motor driving control circuit;
wherein the microcontroller emits a fixed-frequency signal to the motor driving control circuit to provide a fixed drive frequency to the MEMS pump from an initialization stage during the inflation process;
a DC-DC boost circuit boosting a DC voltage of a system power supply to a DC voltage applicable for the motor driving control circuit to provide the DC drive voltage level to the MEMS pump.

2. The blood pressure measurement device of claim 1, wherein the microcontroller controls the MEMS pump to deflate air from the bladder without applying any DC drive voltage to the MEMS pump.

3. The blood pressure measurement device of claim 2, wherein the MEMS pump replaces an air relief unit to release the air from the bladder without applying any DC drive voltage to the MEMS pump.

4. The blood pressure measurement device of claim 1, further comprising a pressure sensor for monitoring the pressure in the bladder during the inflation process.

5. The blood pressure measurement device of claim 1, wherein the microcontroller emits the fixed-frequency signal to the motor driving control circuit to provide the fixed drive frequency to the MEMS pump from the initialization stage till the pressure in the bladder is equal to 150 mmHg during the inflation process.

6. The blood pressure measurement device of claim 1, further comprising an alternating color light source and a transparent or translucent casing covering the MEMS pump, the microcontroller, the DC-DC boost circuit, and the alternating light source, wherein the alternating color light source changes color light with a pulse rate during blood pressure measurement.

7. The blood pressure measurement device of claim 1, further comprising an alternating color light source and a transparent or translucent casing covering the MEMS pump, the microcontroller, the DC-DC boost circuit, and the alternating light source, wherein the alternating color light source changes color between red and green lights to indicate a high blood pressure and a standard blood pressure respectively.

8. The blood pressure measurement device of claim 1, wherein the motor driving control circuit emits a pulse width modulation (PWM) fixed-frequency signal to provide the fixed drive frequency to the MEMS pump.

9. The blood pressure measurement device of claim 1, wherein the DC drive voltage level is increased according to a linear control.

10. The blood pressure measurement device of claim 1, further comprises a flow sensor for detecting the amount of inflation for monitoring the pressure in the bladder.

11. A blood pressure measurement device with a MEMS pump, comprising:
a cuff having a bladder for wrapping around an object to be measured;
a MEMS pump for inflating the bladder with air during an inflation process in a first direction and releasing air from the bladder during a deflation process without using any air relief units, wherein air of the bladder is exhausted through the MEMS pump in a second direction opposite to the first direction; and
a microcontroller controlling a drive voltage level of the MEMS pump so as to maintain a constant air inflation speed of the MEMS pump;
wherein the microcontroller controls the drive voltage level of the MEMS pump by a voltage regulator circuit through a motor driving control circuit;
wherein the microcontroller emits a fixed-frequency signal to the motor driving control circuit to provide a fixed drive frequency to the MEMS pump from an initialization stage during the inflation process;
a DC-DC boost circuit boosting a DC voltage of a system power supply to a DC voltage applicable for the motor driving control circuit to provide the drive voltage level to the MEMS pump.

12. The blood pressure measurement device of claim 11, further comprising a pressure sensor for monitoring the pressure in the bladder during the inflation process.

13. The blood pressure measurement device of claim 11, wherein the microcontroller emits the fixed-frequency signal to the motor driving control circuit to provide the fixed drive frequency to the MEMS pump from the initialization stage till the pressure in the bladder is equal to 150 mmHg during the inflation process.

14. The blood pressure measurement device of claim 11, further comprising an alternating color light source and a transparent or translucent casing covering the MEMS pump, the microcontroller, the DC-DC boost circuit, and the alternating light source, wherein the alternating color light source changes color light with a pulse rate during blood pressure measurement.

15. The blood pressure measurement device of claim 11, further comprising an alternating color light source and a transparent or translucent casing covering the MEMS pump, the microcontroller, the DC-DC boost circuit, and the alternating light source, wherein the alternating color light source changes color between red and green lights to indicate a high blood pressure and a standard blood pressure respectively.

16. A blood pressure measurement device with a MEMS pump, comprising:
a cuff having a bladder for wrapping around an object to be measured;
a MEMS pump for inflating the bladder with air during an inflation process in a first direction when a drive voltage is applied to the MEMS pump and deflating air from the bladder during a deflation process when no drive voltage is applied to the MEMS pump, wherein air of the bladder is exhausted through the MEMS pump in a second direction opposite to the first direction and no air relief unit fluidly communicates with the bladder; and
a microcontroller controlling a drive voltage level of the MEMS pump so as to maintain a constant air inflation speed of the MEMS pump;
wherein the microcontroller controls the drive voltage level of the MEMS pump by a voltage regulator circuit through a motor driving control circuit;
wherein the microcontroller emits a fixed-frequency signal to the motor driving control circuit to provide a fixed drive frequency to the MEMS pump from an initialization stage during the inflation process;
a DC-DC boost circuit boosting a DC voltage from a system power supply to a DC voltage applicable for the motor driving control circuit.

17. The blood pressure measurement device of claim 16, further comprising a pressure sensor for monitoring the pressure in the bladder during the inflation process.

18. The blood pressure measurement device of claim 16, wherein the microcontroller emits the fixed-frequency signal to the motor driving control circuit to provide the fixed drive frequency to the MEMS pump from the initialization stage till the pressure in the bladder is equal to 150 mmHg during the inflation process.

19. The blood pressure measurement device of claim 16, further comprising an alternating color light source and a transparent or translucent casing covering the MEMS pump, the microcontroller, the DC-DC boost circuit, and the alternating light source, wherein the alternating color light source changes color light with a pulse rate during blood pressure measurement.

20. The blood pressure measurement device of claim 16, further comprising an alternating color light source and a transparent or translucent casing covering the MEMS pump, the microcontroller, the DC-DC boost circuit, and the alternating light source, wherein the alternating color light source changes color between red and green lights to indicate a high blood pressure and a standard blood pressure respectively.
